(19) United States
(12) Patent Application Publication
(10) Pub. No.: US 2016/0331465 A1
(43) Pub. Date: Nov. 17, 2016

(54) SYSTEM AND METHOD FOR SIMULATING RECONSTRUCTIVE SURGERY OF ANTERIOR CRUCIATE LIGAMENT USING MEDICAL IMAGES

(71) Applicant: CORELINE SOFT CO., LTD., Seoul (KR)

(72) Inventors: Jin Kook Kim, Seoul (KR); Won Taek Seo, Seongnam (KR); Byeong Soo Kim, Buchon (KR); Hyun Gi Seo, Goyang (KR)

(73) Assignee: CORELINE SOFT CO., LTD.

(21) Appl. No.: 15/153,061

(22) Filed: May 12, 2016

(30) Foreign Application Priority Data
May 12, 2015 (KR) 10-2015-0065752

(51) Int. Cl. A61B 34/10; G06T 7/00; A61B 5/00; A61B 90/00; A61B 5/107

(52) U.S. Cl. CPC A61B 34/10; A61B 90/361; A61B 5/1079; A61B 5/4533; A61B 5/4585; G06T 7/0012; A61B 2034/105; A61B 2034/107; A61B 2090/373; A61B 2576/02; G06T 2207/10012; G06T 2207/30008

(57) ABSTRACT

A system and method for simulating the reconstructive surgery of a cruciate ligament using medical images are disclosed herein. The system includes a processor, the processor is configured to acquire a first image of a knee region of a subject, and a 3D model including the knee region, identify the locations of reference points, on the 3D model, acquire a second image corresponding to the changes in the movement of the knee region, and track changes in the locations of the reference points using the acquired second image, and simulate changes in the length of a virtual ligament attributable to the changes in the movement of the knee region based on the tracked changes in the locations of the reference points and the location of the insertion of the virtual ligament received from a user.

### CROSS-REFERENCE TO RELATED APPLICATION

This application claims under 35 U.S.C. §119(a) the benefit of Korean Application No. 10-2015-0065752 filed on May 12, 2015, which is incorporated herein by reference.

### TECHNICAL FIELD

The present invention relates generally to a system and method for simulating surgery using medical images, and more particularly to a system and method that are capable of simulating the restorative surgery of a cruciate ligament.

### BACKGROUND ART

FIG. 1 is a view showing the structure of a knee joint, and FIG. 2 is a view showing the detailed structure of the knee joint.

Referring to FIGS. 1 and 2, the knee joint includes three bones, i.e., a femur 1, a tibia 3, and a patella 2, and is a hinge joint. The ligaments of the knee joint include a Medial Collateral Ligament (MCL), a Lateral Collateral Ligament (LCL), a Posterior Cruciate Ligament (PCL) 4, and an Anterior Cruciate Ligament (ACL) 5.

Furthermore, the surfaces of the knee joint on which weight load is exerted are covered with articular cartilage. This articular cartilage includes a medial meniscus 6 and a lateral meniscus 7 which are located between the femur and the tibia, and functions to protect the knee joint against the pressure applied between the tibia and the femur and to absorb load exerted on a knee.

In particular, among the ligaments of the knee joint, the ACL 5 is one of the principal ligaments of the knee joint, and functions to aid in enabling the rotation of the knee while preventing the tibia from being dislocated to the front of the femur.

Such an ACL is a region that is most frequently damaged in a knee, and is easily influenced and damaged by an action, such as an abrupt stop, a twist, an erroneous landing, or the like. Generally, it is known that humans who particulate in basketball, soccer, ski, American football or the like have a high possibility of damaging their ACL.

If appropriate treatment is not given even when both an ACL and a PCL have been seriously damaged, this may lead to degenerative arthritis due to the tear of a medial meniscus or the damage of articular cartilage attributable to mid-flexion instability, and thus it is important to give appropriate treatment in early stages.

Methods for treating the damage of an ACL/PCL include a nonsurgical treatment method and a surgical treatment method. In the case of the nonsurgical treatment method, a partial tear having no instability can be restored almost to a state before the damage by giving gradual physical therapy and rehabilitation therapy together with education as conservative treatment. In the case of the surgical treatment method, reconstructive treatment is performed in most cases because the results of a simple suture are not desirable when the simple suture is performed.

In reconstructive treatment that is most frequently given for the tear of an ACL/PCL, a prepared ligament is set to move from a tibia to a femur using a long needle and a thread is tied to the prepared ligament and pulled with the needle, using a method in which a bone tunnel is formed through a tibia and a femur to be disposed at a location identical to the location of a torn ligament and then a ligament is passed through the tunnel. When the ligament is passed through a tibia tunnel and then a femur tunnel and disposed at a predetermined location, the ligament is fastened using various fastening tools. Furthermore, a doctor checks the tension of the implanted ligament, checks whether the range of movement of a knee joint falls within a normal range, evaluates the results of the reconstructive treatment via a knee instability test, and sutures and sterilizes skin, thus resulting in the completion of the reconstructive treatment.

Meanwhile, most types of conventional reconstructive treatment are problematic in that a long surgery time is required because a needle must be passed through a tibia and a femur twice separately in different directions, and are also problematic in that, although surgery is performed after virtual path target spots have been marked on the inside and outside surfaces of a tibia and a femur using an iron in order to prevent the needle from deviating from a correct path when passing through the tibia tunnel and the femur tunnel, the error in which the ligament is not accurately passed through a rectilinear line occurs due to various causes (vibration during the process of holding the femur or the like, the vibration of the hand of the doctor, and the like) and thus accuracy is degraded.

Furthermore, even when it is determined from the point of view of a doctor that reconstructive treatment has been successfully given, there are differences between the knee states of individual patients (i.e., differences in the extent to which an implanted ligament is increased or decreased when a knee is flexed or extended or when the knee is rotated, differences in tension, and the like). Accordingly, in some cases, during walking after surgery, there may occur the instability of a knee joint in which the femur is displaced backward and the tibia is displaced forward as a tibiotarsal joint is flexed and thus gastrocnemius muscles are tensed, or in which the tibia is rotated due to damage.

Accordingly, although in a related field, there is a demand for the development of a technology capable of determining and measuring changes in the state during the period before and after the surgery of a knee joint of a patient in order to reduce the occurrence rate of recurrence, instability, a reconstruction failure and the like that occurs after the reconstructive treatment of a cruciate ligament, an appropriate technology for satisfying the above demand has not been proposed so far.

Meanwhile, Korean Patent Application Publication No. 10-2015-0024982 relates to an apparatus and method for constructing the 3D coordinate system of a knee bone. This patent discloses an apparatus for constructing the 3D coordinate system of a knee bone, including: a photographing unit configured to photograph a 2D image of a femur and a tibia, and to acquire a binary 2D image via a bone segmentation process; and a control unit configured to convert the acquired 2D binary image into a 3D image, to extract an anatomical axis and a landmark central point from the 3D image, and to generate the 3D coordinates of the femur and the tibia using the extracted anatomical axis and landmark central point.

However, the preceding technology focuses merely on a technology for extracting coordinate points based on a 3D image of a knee bone in order to measure changes in cartilage during the period before and after arthritis surgery. Therefore, this preceding technology cannot yet overcome the problems of the conventional technology in that the error in which a ligament is not accurately passed through a rectilinear line occurs due to various causes (vibration during the process of holding a femur or the like, the vibration of the hand of a doctor, and the like) during reconstructive surgery or in that recurrence, instability and the like that may occur later cannot be prevented in advance because differences between the knee states of individual patients (for example, differences in the extent to which an implanted ligament is increased or decreased when a knee is flexed or extended or when the knee is rotated, differences in tension, and the like) are not taken into account.

### SUMMARY OF THE DISCLOSURE

An object of the present invention is to provide a system and method for simulating surgery using medical images, particularly a system and method that are capable of simulating the restorative surgery of a cruciate ligament.

An object of the present invention is to reduce the occurrence rate of recurrence, instability, a reconstruction failure and the like that occur after the reconstructive treatment of a cruciate ligament.

An object of the present invention is to simulate changes in the length of a cruciate ligament when a knee moves.

An object of the present invention is to provide the most appropriate location where a ligament will be inserted by taking into account the state of knee movement of a patient (i.e., a difference in the extent to which an implanted ligament is increased or decreased when a knee is flexed or extended or when the knee is rotated, a difference in tension, and the like).

An object of the present invention is to provide convenience to the restorative surgery of a cruciate ligament.

An object of the present invention is to acquire information about the actual movement of a knee of a patient based on a medical image acquired by photographing the actual movement of the knee of the patient and then simulate changes in the length of a cruciate ligament attributable to the actual movement of the knee of the patient.

In accordance with an aspect of the present invention, there is provided a method for simulating the reconstructive surgery of a cruciate ligament using medical images executed by a processor within a computing system, the method including: acquiring a first image of a knee region of a subject on which ligament reconstructive surgery will be performed, and a 3D model which is generated to include the knee region; identifying the locations of reference points, which are references for tracking changes in the movement of the knee region, on the 3D model; acquiring a second image corresponding to the changes in the movement of the knee region; tracking changes in the locations of the reference points, identified on the 3D model, using the acquired second image corresponding to the changes in the movement; and simulating changes in the length of a virtual ligament attributable to the changes in the movement of the knee region based on the tracked changes in the locations of the reference points and the location where the virtual ligament is inserted received from a user. The first image of the knee region is an image including the anatomical structure of the knee region.

The identifying may include identifying the first locations of the reference points in the acquired second image corresponding to the changes in the movement, and then identifying the locations of the reference points on the 3D model corresponding to the first locations of the reference points, by registering the second image with the 3D model. The identifying may include identifying the locations of the reference points by extracting the locations of feature points relative to a bone region or a rigid body region on the 3D model, or identifying the locations of the reference points based on marking information received from the user. The method may further include displaying the results of the changes in the length of the virtual ligament attributable to the simulation. The displaying may include visualizing changes in the length of a first portion of the virtual ligament attributable to the changes in the movement of the knee region, the first portion is inserted into a first bone, visualizing changes in the length of a second portion of the virtual ligament attributable to the changes in the movement of the knee region, the second portion is inserted into a second bone, and visualizing changes in the length of a third portion of the virtual ligament attributable to the changes in the movement of the knee region, the third portion is located between the first and second portions The displaying may include displaying a first portion of the virtual ligament, inserted into a first bone, in a first color, displaying a second portion of the virtual ligament, inserted into a second bone, in a second color, and displaying a third portion of the virtual ligament, located between the first and second portions, in a third color.

The method may further include displaying the location of the insertion of the virtual ligament, at which the results of the changes in the length of the virtual ligament attributable to the simulation are minimized, as a recommended insertion location.

In accordance with another aspect of the present invention, there is provided a method for simulating the reconstructive surgery of a cruciate ligament using medical images, the method including: receiving at least one location where a virtual ligament is inserted from a user on a previously stored image corresponding to changes in the movement of a knee region of a subject on which ligament reconstructive surgery will be performed; simulating changes in the length of the virtual ligament attributable to the changes in the movement of the knee region based on the at least one location input of the user; and displaying the changes in the length of the virtual ligament attributable versus at least one location of the insertion of the virtual ligament received from the user using the results of the simulation.

The receiving may include receiving the location where the virtual ligament is inserted relative to a bone region or a rigid body region on a previously stored 3D model of the knee region as locations of reference points, which are references for tracking the changes in the length of the virtual ligament attributable to the changes in the movement of the knee region. The displaying may include displaying a graph representative of, or visualizing, changes in the length of each of a first portion inserted into a first bone, a second portion inserted into a second bone, and a third portion located between the first and second portions, which are included in the virtual ligament. The displaying may include displaying a first portion inserted into a first bone, a second portion inserted into a second bone, and a third portion located between the first and second portions based on the first and second bones in different colors.

The method may further include displaying a list of recommended insertion locations calculated based on the simulation results of the changes in the length of the virtual ligament attributable to the simulation.

In accordance with still another aspect of the present invention, there is provided a system for simulating the reconstructive surgery of a cruciate ligament using medical images, the system including a processor, the processor includes: an image acquisition unit configured to acquire a first image of a knee region of a subject on which ligament reconstructive surgery will be performed, and a 3D model which is generated to include the knee region; an identification unit configured to identify the locations of reference points, which are references for tracking changes in the movement of the knee region, on the 3D model; a tracking unit configured to acquire a second image corresponding to the changes in the movement of the knee region, and to track changes in the locations of the reference points, identified on the 3D model, using the acquired second image corresponding to the changes in the movement; and a simulation unit configured to simulate changes in the length of a virtual ligament attributable to the changes in the movement of the knee region based on the tracked changes in the locations of the reference points and based on the location where the virtual ligament is inserted received from a user.

The identification unit may be further configured to identify the first locations of the reference points in the acquired second image corresponding to the changes in the movement, and to then identify the locations of the reference points on the 3D model corresponding to the first locations of the reference points by registering the second image with the 3D model.

The identification unit may be further configured to identify the locations of the reference points by extracting the locations of feature points relative to a bone region or a rigid body region on the 3D model, or to identify the locations of the reference points based on marking information received from the user.

The system may further include a display control unit configured to display the results of the changes in the length of the virtual ligament attributable to the simulation. The display control unit may be further configured to display a graph representative of, or visualize, changes in the length of each of a first portion inserted into a first bone, a second portion inserted into a second bone, and a third portion located between the first and second portions based on the first and second bones, which are included in the virtual ligament. The display control unit may be further configured to display the location of where the virtual ligament is inserted, at which the results of the changes in the length of the virtual ligament attributable to the simulation are minimized, as a recommended insertion location.

In accordance with still another aspect of the present invention, there is provided a system for simulating reconstructive surgery of a cruciate ligament using medical images, the system including a processor, the processor includes: an interface control unit configured to receive the at least one location where a virtual ligament will be inserted from a user on a previously stored image corresponding to changes in the movement of a knee region of a subject on which ligament reconstructive surgery will be performed; a simulation unit configured to simulate changes in the length of the virtual ligament attributable to the changes in the movement of the knee region based on the at least one location of the insertion of the virtual ligament input of the user; and a display control unit configured to display the results of the changes in the length of the virtual ligament versus the at least one location of the virtual ligament input from the user attributable to the simulation.

The interface control unit may be further configured to receive the location where the virtual ligament is inserted relative to a bone region or a rigid body region on a previously stored 3D model of the knee region as the locations of reference points, which are references for tracking the changes in the length of the virtual ligament attributable to the changes in the movement of the knee region. The display control unit may be further configured to display a list of recommended insertion locations calculated based on the simulation results of the changes in the length of the virtual ligament versus the at least one location of the insertion of the virtual ligament input from the user attributable to the simulation.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view showing the structure of a knee joint;

FIG. 2 is a view showing the detailed structure of the knee joint;

FIG. 3 is a diagram showing a first configuration of a system for simulating the reconstructive surgery of a cruciate ligament using medical images according to an embodiment of the present invention;

FIG. 4 is a diagram showing a second configuration of a system for simulating the reconstructive surgery of a cruciate ligament using medical images according to an embodiment of the present invention;

FIG. 5 is a diagram showing a third configuration of a system for simulating the reconstructive surgery of a cruciate ligament using medical images according to an embodiment of the present invention;

FIG. 6 is a view showing an image of a knee region acquired by an image acquisition unit according to an embodiment of the present invention;

FIG. 7 is a view showing an example of the identification of reference points according to an embodiment of the present invention;

FIG. 8 is a view showing examples of the display of an image including reference points according to an embodiment of the present invention;

FIGS. 9A, 9B, and 9C are views showing examples of movement that is performed to acquire an image corresponding to changes in the movement of a knee region according to an embodiment of the present invention;

FIG. 10 is a view showing an example of the display of an inserted virtual ligament according to an embodiment of the present invention;

FIG. 11 is a view showing an example of the display of a graph representative of changes in the length of a ligament according to an embodiment of the present invention;

FIG. 12 is a view showing an example of the location of the insertion of a virtual ligament received from a user according to an embodiment of the present invention;

FIG. 13 is a first operation flowchart of a method for simulating the reconstructive surgery of a cruciate ligament using medical images according to an embodiment of the present invention; and

FIG. 14 is a second operation flowchart of a method for simulating the reconstructive surgery of a cruciate ligament using medical images according to an embodiment of the present invention.

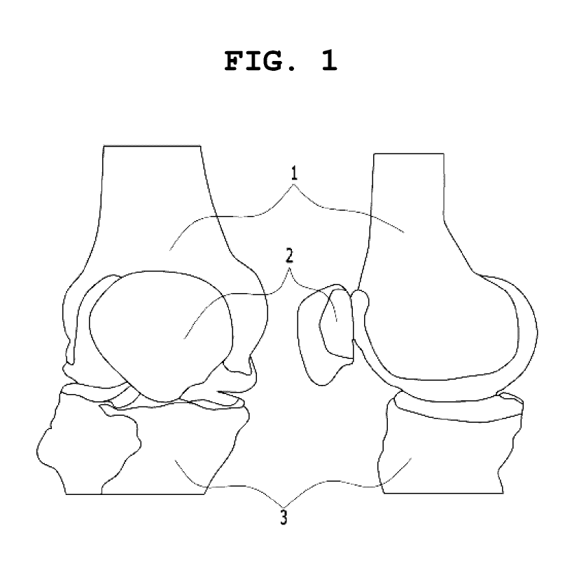

### DETAILED DESCRIPTION OF THE DISCLOSURE

Embodiments of the present invention will be described in detail below with reference to the accompanying drawings. In the following description, detailed descriptions of related known components or functions that may unnecessarily make the gist of the present invention obscure will be omitted. Furthermore, in the following description of embodiments of the present invention, specific numerical values are merely examples.

The present invention relates generally to a system and method for simulating surgery using medical images, and more particularly to a system and method that are capable of simulating the restorative surgery of a cruciate ligament. According to the present invention, a ligament can be virtually inserted and then changes attributable to the movement of a knee (i.e., changes in the length of the ligament, changes in the tension of the ligament, etc. when the knee moves) can be simulated before the reconstructive surgery of a cruciate ligament is actually performed. Accordingly, using this, the advantage of finding the most appropriate location into which a ligament will be inserted and the advantage of reducing the occurrence rate of recurrence, instability, a reconstruction failure and the like that occur after the reconstructive surgery of a cruciate ligament can be achieved.

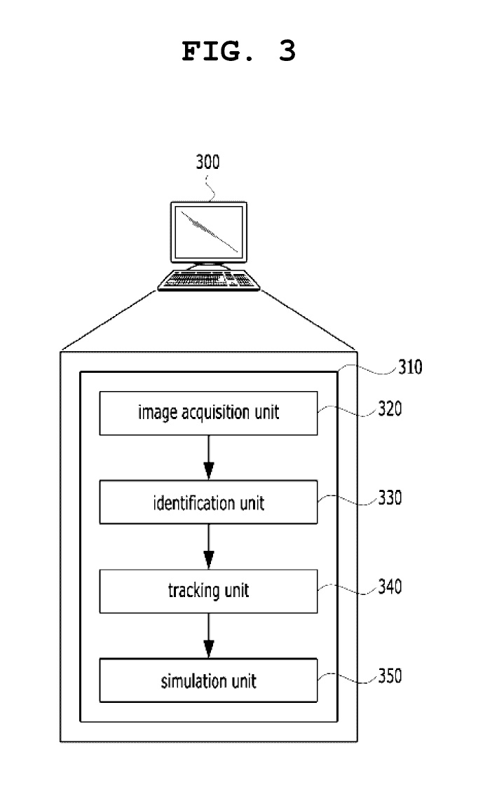

FIG. 3 is a diagram showing a first configuration of a system for simulating the reconstructive surgery of a cruciate ligament using medical images according to an embodiment of the present invention.

Referring to FIG. 3, a system 300 for simulating the reconstructive surgery of an ACL using medical images according to an embodiment of the present invention may include a processor 310, and the processor 310 may include an image acquisition unit 320, an identification unit 330, a tracking unit 340, and a simulation unit 350.

The image acquisition unit 320 acquires an image of a knee region of a subject on which the restoration (or reconstruction) surgery of a ligament will be performed, and a three-dimensional (3D) model (in particular, a 3D anatomical model) which is generated to include the knee region.

In this case, the image of the knee region of the subject acquired by the image acquisition unit 320 may be an image of the knee region before the insertion of a ligament in order to virtually simulate the insertion of the ligament before a doctor actually inserts the ligament into a knee of the subject for the restorative surgery of the ligament, and the image of the knee region may be an image including thigh and calf regions.

Furthermore, the image acquisition unit 320 may acquire a 2D image, including a femur, a tibia and a patella, as a first image, i.e., the image of the knee region.

Furthermore, the processor 310 according to an embodiment of the present invention may include a modeling unit (not shown) in order to acquire a 3D model generated to include a knee region, and the modeling unit may generate a 3D anatomical model based on the image of the 2D knee region acquired from the image acquisition unit 320. In this case, the 3D model generated to include the knee region may model not only a femur, a tibia and a patella but also an MCL, an LCL, a PCL and an ACL in detail. The 3D model may also model articular cartilage, such as a medial meniscus and a lateral meniscus, in detail.

Furthermore, the image acquisition unit 320 may acquire a second image (i.e., a moving image) corresponding to changes in the movement of the knee region. The processor 310 according to an embodiment of the present invention may store the first image of the knee region, the 3D model generated to include the knee region, and the second image corresponding to the changes in the movement of the knee region, acquired by the image acquisition unit 320, in a database (not shown). The second image may be captured when the knee region is actually moving, for example, forward and backward, marker or any identifiable point is attached to the tibia and/or femur.

The identification unit 330 identifies the locations of a plurality of reference points, i.e., references for tracking the changes in the movement of the knee region, on the 3D model.

That is, the present invention is a technology that is capable of virtually simulating the results of the insertion of a ligament in order to insert the ligament into a location at which a change in the length of the ligament is minimized when a corresponding knee moves after the restorative surgery of the ligament. In order to find the appropriate location of the insertion of a ligament at which a change in the length of the ligament is minimized, it is necessary to track the movement of the knee related to changes in the length of the ligament when the knee moves. In other words, the present invention requires the locations of reference points, i.e., references for tracking changes in the movement of a knee, in order to find the location of the insertion of a ligament at which a change in the length of the ligament is minimized. Accordingly, the identification unit 330 may identify the locations of reference points, i.e., references, on a 3D model of a knee region.

In this case, the identification unit 330 may identify the locations of reference points by automatically extracting the locations of the feature points through image processing based on the 3D model, particularly a bone region or a rigid body region, or may identify the locations of reference points based on marking information input onto the 3D model by a user. Furthermore, the identification unit 330 may identify the locations of reference points on the 3D model through image registration after the user has actually attached or installed markers onto a knee bone or skin of a patient. This will be described in greater detail later.

Furthermore, the location of a single reference point may be identified on each of a femur and a tibia. Alternatively, a plurality of reference points may be identified on each of a femur and a tibia in order to enhance the reliability of simulation based on the tracking of the locations of reference points. In this case, the more collaborate results of simulation regarding the movement of a knee may be acquired by tracking changes in the relative locations between the plurality of reference points on the femur and the plurality of reference points on the tibia.

When the reference points are markers actually attached or installed onto a knee region of a patient, the identification unit 330 may identify the locations of the reference points in the second image corresponding to changes in the movement acquired by the image acquisition unit 320, may register the second image with the 3D model, and then may identify locations corresponding to the locations of the reference points, identified in the second image, on the 3D model.

In greater detail, as an example, the following process may be performed in order to identify the locations of reference points on a 3D model.

First, a doctor, i.e., an expert, may actually attach or install markers, which may be seen from the moving image, onto the knee bone or skin of a patient, and then may move the knee of the patient by holding and forcibly moving a corresponding leg of the patient. Thereafter, after a moving image of the movement of the knee of the patient (i.e., an image corresponding to changes in the movement of the knee region) has been acquired by the image acquisition unit 320, the identification unit 330 primarily identifies the locations of the markers in the acquired moving image. Thereafter, the identification unit 330 may register the acquired moving image with the 3D model in order to determine the locations of the identified markers on the 3D model. Thereafter, the identification unit 330 may identify locations corresponding to the locations of the identified markers on the 3D model, and then the tracking unit 340 may track changes in the locations of the reference points using the locations identified on the 3D model, i.e., the locations of the reference point identified on the 3D model.

That is, the gist of the present invention is to find a location at which a change in the length of a ligament is minimized when a knee moves, as described above. Accordingly, the locations of the reference points, i.e., references for tracking changes in the movement of the knee region, identified by the identification unit 330 may be identified by automatically extracting them as feature points on the 3D model through image processing. Furthermore, according to another embodiment of the present invention, the locations of reference points may be identified based on marking information input onto the 3D model by the user. In this case, in an embodiment of the present invention, the reference points may be input onto a bone region or a rigid body region on the 3D model by the user. In this case, the locations of the reference points may be identified on a 3D model of a bone, such as a bone region or a rigid body region, i.e., a solid structure constituting part of a knee joint, particularly a femur, a tibia, or a patella. Furthermore, according to still another embodiment of the present invention, the locations of reference points regarding markers may be primarily identified on an image of the movement of a knee (i.e., a second image) using knee markers actually attached or installed onto a bone or skin of a patient, and then may be secondarily identified on a 3D model through image registration, thereby identifying the locations of the reference points on the 3D model.

The tracking unit 340 acquires an image corresponding to changes in the movement of the knee region (which is the second image, and refers to a moving image acquired by photographing the movement of the knee region) (in this case, the image corresponding to the changes in the movement of the knee region may be acquired by the acquisition unit 320), and tracks changes in the locations of the reference points identified on the 3D model using the acquired image corresponding to the changes in the movement.

In this case, in an embodiment of the present invention, in order to acquire the image corresponding to the changes in the movement of the knee region via the tracking unit 340, a process in which a corresponding knee of a patient is actually moved by a skilled doctor, as described above, may be performed after or before the identification unit 330 identifies the locations of the reference points. Furthermore, in this case, the knee of the patient may be moved according to his or her own will, or may be moved in such a way that a doctor, i.e., an expert, holds a corresponding leg of the patient and forcibly moves the knee.

Furthermore, in this case, during the process in which the knee is moved by the doctor or patient, knee bending movement, i.e., flexion movement and extension movement, may be performed, or knee twisting movement may be performed. The actual movement of a knee region of a patient is considerably complicated movement achieved by the combination of rotating movement and sliding movement, and may not be easily modeled because it varies with his or her gait or the form of his or her knee joint. In an embodiment of the present invention, image information about the movement of the knee joint during actual movement may be acquired for each patient by tracking changes in relative locations between reference points located on a tibia and a femur in response to the actual movement of the knee.

That is, the tracking unit 340 acquires an image corresponding to changes in the movement of the knee region (i.e., a moving image acquired by photographing the bending movement of the knee) as the knee joint of the patient is actually subjected to bending movement, and tracks changes in the locations of the reference points identified by the identification unit 330 using the acquired image corresponding to the changes in the movement. In particular, the tracking unit 340 may track changes in the locations of the reference points when the knee joint of the patient actually moves (i.e., the knee joint is subjected to bending movement, sliding movement, and rotating movement) using the locations of the reference points identified on the 3D model through image registration.

The simulation unit 350 simulates changes in the length of a virtual ligament attributable to the changes in the movement of the knee region based on the changes in the location of the reference points tracked by the tracking unit 340 and based on the location of the insertion of the virtual ligament received from the user. The processor 310 according to an embodiment of the present invention holds 3D models of the surfaces of the femur and tibia of the knee of the patient based on previously acquired medical images, and thus a motion profile of the 3D model of the knee during actual movement may be acquired as the results of simulation by tracking changes in the relative locations between the reference points and applying the results of the tracking to the 3D model.

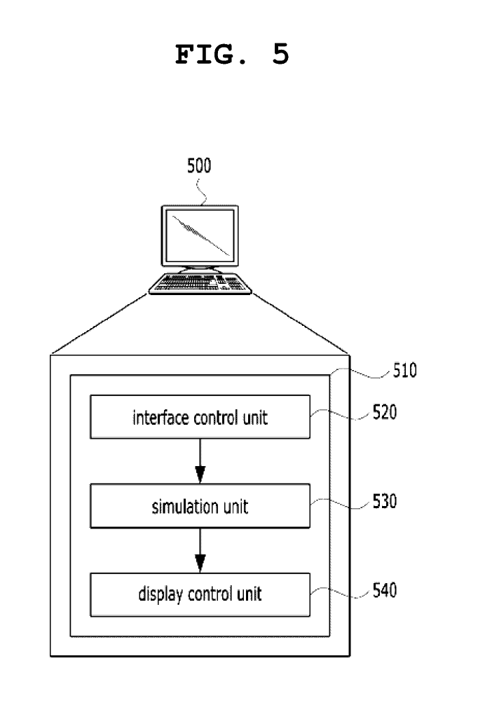

In this case, in an embodiment of the present invention, a location into which the virtual ligament will be inserted (i.e., the location of the insertion of the virtual ligament on which simulation will be performed) may be received from the user via an interface control unit 520 (which will be described later in conjunction with FIG. 5), and the location into which the virtual ligament will be inserted may be determined through the input of the user (in this case, the input of the user may be an input, such as a click or a drag, input onto the 3D model using a mouse or the like, and may include one or more inputs) by taking into account a portion where a change in the location of the reference point tracked by the tracking unit 340 is small.

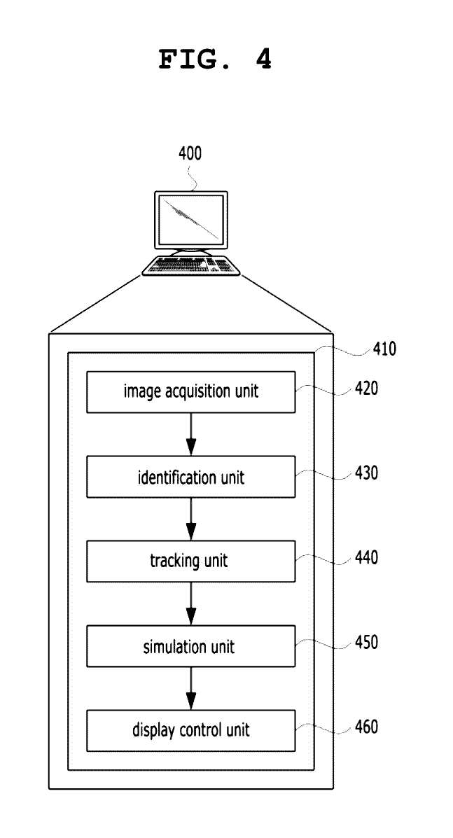

Alternatively, the location of the insertion of the virtual ligament may be automatically calculated by the system 300 for a location at which there is no change in the location or a change in the location is minimum based on the changes in the locations of the reference points tracked by the tracking unit 340, and one or more recommendation lists of the calculated locations of the insertion of the virtual ligament may be displayed on a screen by a display control unit 460 (which will be described later in conjunction with FIG. 4). In this case, thereafter, the user may select at least one from the recommendation lists of the locations of the insertion of the virtual ligament displayed on the screen, the interface control unit 520 may identify the corresponding list selected by the user among the recommendation lists, and then the simulation unit 350 may perform simulation on the location of the insertion of the virtual ligament corresponding to the identified corresponding list.

Meanwhile, the ligament inserted for the restorative surgery of a ligament may be divided into a total of three portions, i.e., a ligament located inside a femur, a ligament located inside a tibia, and a ligament adapted to connect the ligaments located inside the femur and the tibia (i.e., a ligament exposed to the outside, other than the ligaments located inside the femur and the tibia). In an embodiment of the present invention, for ease of description, the femur is referred to as a first bone, the tibia is referred to as a second bone, a virtual ligament inserted into the first bone for simulation is referred to as a first portion, a virtual ligament inserted into the second bone is referred to as a second portion, and a virtual ligament located between the first and second bones and exposed to the outside is referred to as a third portion. This is merely an embodiment, and the present invention is not limited thereto. In another embodiment, the tibia may be a first bone, and the femur may be a second bone.

Accordingly, when receiving the location of the insertion of the virtual ligament from the user, the interface control unit 520 may receive the start and end points of the first portion and the start and end points of the second portion, and the location of the insertion of the third portion may be automatically determined based on the start point or end point received for the first and second portions.

That is, in an embodiment of the present invention, the location of the insertion of the virtual ligament may be determined using marking information regarding the location of the insertion of the ligament input onto the 3D model, particularly the first bone or the second bone, received from the user, or may be determined using a list of the recommended locations of the insertion of the ligament automatically calculated by the system 300.

The simulation unit 350 simulates changes in the length of the virtual ligament attributable to changes in the movement of the knee region based on the location of the insertion of the virtual ligament received from the user. In this case, the simulation unit 350 may simulate changes in the length of each of the first to third portions.

The simulation unit 350 according to an embodiment of the present invention may simulate how the inserted ligament changes when the knee is subjected to bending movement (in particular, how the length of the inserted ligament changes, the extent of tension, and the like), and may find a location at which a change in the length of the ligament is minimized based on the results of the simulation. Furthermore, according to an embodiment of the present invention, the restorative surgery of a ligament is actually performed based on the results of the simulation, thereby reducing the probabilities of recurrence, instability, and the failure of reconstruction that may occur after surgery. That is, the present invention can improve the success rate of the restorative surgery of cruciate ligaments.

FIG. 4 is a diagram showing a second configuration of a system for simulating the reconstructive surgery of a cruciate ligament using medical images according to an embodiment of the present invention.

Referring to FIG. 4, a system 400 for simulating the reconstructive surgery of an ACL using medical images according to another embodiment of the present invention may include a processor 410, and the processor 410 may include an image acquisition unit 420, an identification unit 430, a tracking unit 440, a simulation unit 450, and a display control unit 460.

In this case, since the processor 410, image acquisition unit 420, identification unit 430, tracking unit 440 and simulation unit 450 of FIG. 4 are the same as the processor 310, the image acquisition unit 320, the identification unit 330, the tracking unit 340, and the simulation unit 350 shown in FIG. 3, respectively, redundant descriptions thereof are omitted below.

Referring to FIG. 4, the display control unit 460 displays the results of changes in the length of a virtual ligament attributable to simulation on a screen. That is, once the simulation unit 450 has performed a simulation of the location of the insertion of the virtual ligament, the display control unit 460 displays the results of the simulation on the screen.

In this case, the display control unit 460 may display a graph representative of changes in the length of each of a first portion inserted into a first bone, a second portion inserted into a second bone, and a third portion located between the first and second portions based on the first and second bones, which are included in the virtual ligament based on the location of the insertion of the virtual ligament received from a user, as the results of the changes in the length of the virtual ligament. That is, with regard to the first portion, i.e., a virtual ligament inserted into first bone (a femur), the second portion, i.e., a virtual ligament inserted into the second bone (a tibia), and the third portion, i.e., a virtual ligament located between the first and second bones and exposed to the outside, the display control unit 460 may display changes in the length of each of the first to third portions in the form of a graph. Using the displayed graph, the user (a doctor) may determine a location into which the ligament is inserted and at which a change in the length is minimized through comparison between a plurality of locations.

The display control unit 460 may provide information about changes in the length of each of the first to third portions in the form of a graph or in the form of numerical values. Furthermore, the display control unit 460 may display the first to third portions on the 3D model in different colors so that the user can easily identify the extent of changes in the length of each of the first to third portions.

Furthermore, the display control unit 460 may display the location of the insertion of the virtual ligament at which the results of a change in the length of the virtual ligament attributable to the simulation are minimized as a recommended insertion location. That is, the present invention aims to find the location of the insertion of a ligament at which a change in the length of the ligament is minimized when a knee moves after surgery in order to overcome inconvenience or instability that the user feels during walking or during exercise because the ligament is excessively tight or loose after the restorative surgery of the ligament. In the graph displayed on the screen by the display control unit 460, the location of the insertion of the ligament at which changes in the lengths of the first to third portions are minimized may be displayed in the form of a separate indication (for example, a recommended insertion location is displayed on a graph in a thick color, a different color, or the like) as the appropriate location of the insertion of the ligament during actual surgery.

In this case, the display control unit 460 may display information about one or more recommended insertion locations as recommended insertion locations, and information about the set number of recommended insertion locations may be previously received from the user and then set. For example, once the user has previously set the number of recommended insertion locations to 3, the display control unit 460 may provide three highest-rank locations at which the results of the changes in the length of the virtual ligament attributable to the simulation are minimized as a list of the recommended locations of the insertion of the ligament. Furthermore, the display control unit 460 may display numerical value information regarding the recommended insertion locations on a graph in an overlay manner.

FIG. 5 is a diagram showing a third configuration of a system for simulating the reconstructive surgery of a cruciate ligament using medical images according to an embodiment of the present invention.

Referring to FIG. 5, a system 500 for simulating the reconstructive surgery of an ACL using medical images according to an embodiment of the present invention may include a processor 510, and the processor 510 may include an interface control unit 520, a simulation unit 530, and a display control unit 540.

In this case, since the processor 510, simulation unit 530 and display control unit 540 of FIG. 5 are the same as the processor 310 or 410, the simulation unit 350 or 450, and the display control unit 460 shown in FIG. 3 or 4, respectively, a brief description is given below based on the above-described details.

The interface control unit 520 receives the location of the insertion of a virtual ligament from a user based on a previously stored image corresponding to changes in the movement of a knee region of a subject on which the restorative surgery of a ligament will be performed.

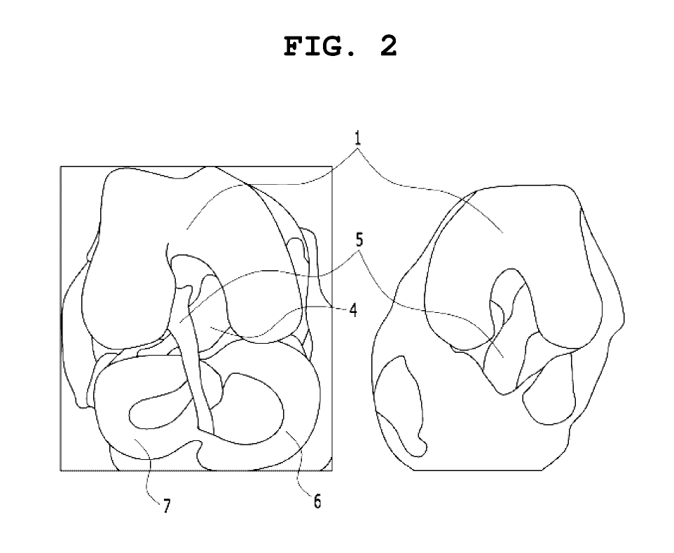

In this case, information about the previously stored image may include not only the image (i.e., a moving image) corresponding to the changes in the movement of the knee region of the subject on which the restorative surgery of a ligament will be performed, but also an image (a 2D image) of the knee region and an image of a 3D model generated to include the knee region. This image information may be previously stored in the image acquisition unit 320 or 420 shown in FIG. 2 or 3.

The interface control unit 520 may receive the location of the insertion of the virtual ligament from the user on the image corresponding to the changes in the movement of the knee region or on the 3D model. Furthermore, the interface control unit 520 may receive the location of the insertion of the virtual ligament through a mouse click or drag. In this case, user input may include one or more inputs.

The interface control unit 520 may receive the location of the insertion of the virtual ligament relative to a bone region or a rigid body region on a previously stored 3D model of the knee region as a plurality of reference points, i.e., references for tracking the changes in the length of the virtual ligament attributable to the changes in the movement of the knee region (i.e., in this case, the reference points refer to points that are references for a location into which the virtual ligament will be inserted). That is, when the user marks a plurality of points on the 3D model, particularly a bone region or a rigid body region, the interface control unit 520 may identify the plurality of marking points marked on the bone region or rigid body region as reference points, i.e., references for tracking changes in the length of the virtual ligament, and then the simulation unit 530 may simulate changes in the length of the virtual ligament attributable to the changes in the movement of the knee region based on the identified reference points.

Meanwhile, as an example, when receiving the location of the insertion of the virtual ligament from the user, the interface control unit 520 may receive the start and end points of a first portion and the start and end points of a second portion via mouse clicks. In this case, the location of the insertion of the third portion may be automatically determined based on the start point or end point received for the first and second portions.

In an embodiment of the present invention, the location of the insertion of the virtual ligament may be determined using marking information regarding the location of the insertion of the ligament input onto the 3D model, particularly a first bone or a second bone, received from the user, or may be determined using candidate lists of the locations of the insertion of the ligament automatically calculated by the system 500. Since this has been described in detail above, a description thereof is omitted below.

The simulation unit 530 simulates changes in the length of the virtual ligament attributable to the changes in the movement of the knee region based on the input of the user. In this case, the simulation unit 530 may simulate changes in the length of each of the first portion, the second portion and the third portion.

The display control unit 540 may display the results of the changes in the length of the virtual ligament attributable to the simulation. In this case, the display control unit 540 may display a graph representative of changes in the length of each of the first to third portions of the virtual ligament as the results of the simulation. Furthermore, the display control unit 540 may display the first to third portions on the 3D model in different colors so that the user can easily identify the extent of changes in the length of each of the first to third portions. Since this has been described in detail above, a description thereof is omitted below.

Furthermore, the display control unit 540 may display a list of recommended insertion locations calculated based on the results of the changes in the length of the virtual ligament attributable to the simulation. In this case, the display control unit 540 may calculate an insertion location at which a change in the length of the virtual ligament is minimized as the most appropriate location of the insertion of the ligament, and may display the calculated location information on a screen.

Furthermore, the display control unit 540 may provide only information about a location at which a change in the length of the ligament is minimized, i.e., only one piece of information, as the list of recommended insertion locations, or may provide a plurality of pieces of information in the form of a list according to a criterion previously set by the user. For example, once the user has previously set the number of recommended insertion locations to 3, the display control unit 540 may provide three highest-rank locations at which the results of the changes in the length of the virtual ligament attributable to the simulation are minimized as a list of the recommended locations of the insertion of a ligament. Furthermore, the display control unit 540 may display numerical value information corresponding to a recommended insertion location on a graph as detailed information regarding the recommended insertion location in an overlay manner.

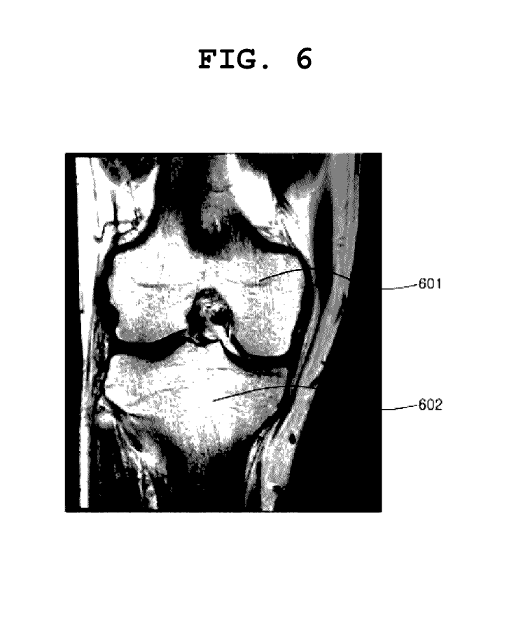

FIG. 6 is a view showing an image of a knee region acquired by an image acquisition unit according to an embodiment of the present invention.

Referring to FIG. 6, the image acquisition unit 320 or 420 according to the embodiment of the present invention acquires a 2D image of a knee region. The 2D image of a knee region may be an x-ray image of the knee region or the like. Furthermore, it can be seen that a femur 601 is located in the upper portion of the image of the knee region acquired by the image acquisition unit 320 or 420 as a first bone and a tibia 602 is located in the lower portion of the image of the knee region as a second bone.

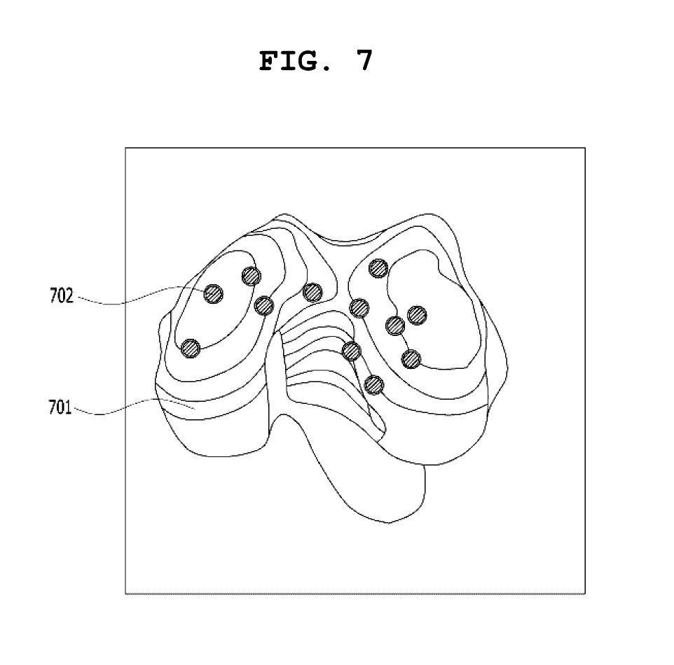

FIG. 7 is a view showing an example of the identification of reference points according to an embodiment of the present invention.

Referring to FIG. 7, in the embodiment of the present invention, a 3D anatomical model is modeled based on a 2D image acquired by the image acquisition unit 320 or 420 prior to identification of the reference points, and the reference points are identified based on the 3D model of a knee region.

In this case, when identifying the locations of reference points, i.e., references for tracking changes in the movement of the knee region, the identification unit 330 or 430 may identify the locations of the reference points by automatically extracting the feature points on a 3D model, particularly a bone region or a rigid body region, through image processing (in this case, the feature points automatically extracted and identified through image processing may be viewed as reference points), or by identifying the reference points based on marking information input onto a 3D model by a user.

Furthermore, the identification unit 330 or 430 may primarily identify the locations of the reference points in a second image (i.e., an image corresponding to changes in the movement of the knee region) via markers actually attached or installed onto a knee bone or skin of a patient, and may identify locations corresponding to the identified locations of the reference points in the second image on the 3D model through the registration of the second image with the 3D model. That is, the identification unit 330 or 430 may identify the locations of the reference points on the 3D model using image registration.

That is, the reference points are points, i.e., references for tracking the changes in the movement of the knee region. As shown in FIG. 7, a plurality of points 702 may be indicated on a first bone 701, i.e., a femur. These points may be automatically extracted and indicated on a 3D model through image processing, may be indicated through the registration of the second image with the 3D model, or may be indicated based on marking information received from the user.

In this case, since the gist of the present invention is to find a location at which a change in the length of a ligament is minimized when a knee moves as described above, reference points for tracking changes in the movement of the knee may be identified on a structure constituting part of a knee joint, i.e., a bone region or a rigid body region (in particular, a bone such as a femur or a tibia).

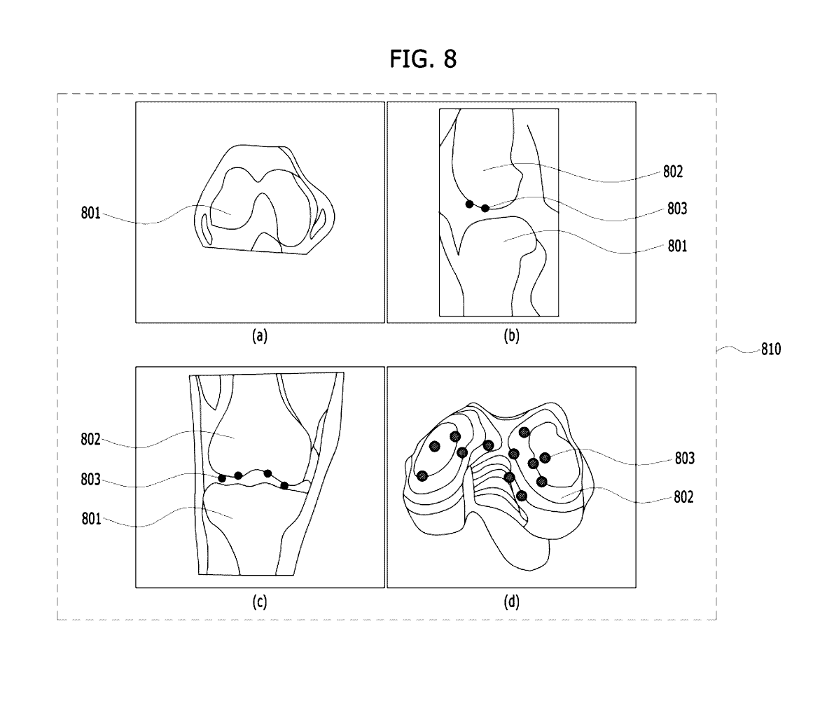

FIG. 8 is a view showing examples of the display of an image including reference points according to an embodiment of the present invention.

Referring to FIG. 8, the display control unit 460 or 540 may display a plurality of images of a knee region, photographed from various angles, together on a single screen 810.

As an example, (a) of FIG. 8 shows a tomographic image of a second bone 801 photographed using Computed Tomography (CT) (in this case, not only a CT image but also an MRI magnetic resonance imaging image may be displayed), (b) of FIG. 8 shows a side image of a knee region, (c) of FIG. 8 is a front image of the knee region, and (d) of FIG. 8 shows a 3D model image of a first bone 802.

Furthermore, a reference point 803 indicated on the 3D model of (d) of FIG. 8 may be indicated on the 2D images of (a) to (c) of FIG. 8 at a location corresponding to the location of the reference point 803 indicated in (d) of FIG. 8.

Furthermore, the system 300, 400 or 500 according to the embodiment of the present invention may include a database (not shown), and the database may store not only an image acquired by the image acquisition unit 320 or 420 but also images on which a reference point has been indicated. That is, referring to FIG. 8, a 2D image in which the reference point 803 has been indicated (for example, the image of (b) or (c) of FIG. 8) and a 3D image on which the reference point 803 has been indicated (for example, the image of (d) of FIG. 8) may be stored in the database.

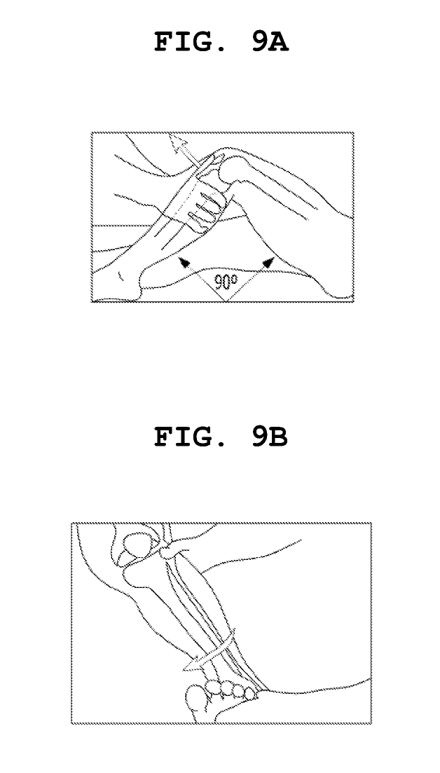

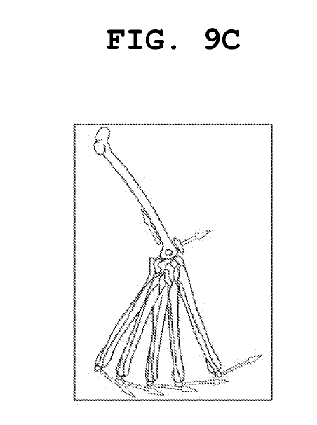

FIGS. 9A, 9B, and 9C are views showing examples of movement that is performed to acquire an image corresponding to changes in the movement of a knee region according to an embodiment of the present invention.

Referring to FIGS. 9A, 9B, and 9C, the tracking unit 340 or 440 according to the embodiment of the present invention acquires an image corresponding to changes in the movement of a knee region (i.e., a moving image acquired by photographing the movement of the knee region), and tracks changes in the location of a reference point identified on a 3D model using the acquired image corresponding to the changes in the movement. In this case, in order to acquire the image corresponding to the changes in the movement of the knee region, a process in which a skilled doctor actually moves a knee of a patient may be performed after the reference point has been identified by the identification unit 330 or 430.

That is, according to an embodiment of the present invention, in order to track changes in the location of a reference point attributable to the movement of a knee region, a doctor or a patient himself or herself may perform flexion movement and extension movement after the reference point has been identified. Alternatively, after the reference point has been extracted, anterior and posterior translation movement may be performed as shown in FIG. 9A, and pivot movement adapted to rotate a joint around an axis may be performed as shown in FIG. 9B. FIG. 9C shows an example of the acquisition of an image corresponding to changes in the movement of an ankle region. The image of FIG. 9C may be an image that is acquired when the pivot movement of an ankle is performed as shown in FIG. 9B.

The acquisition unit 320 or 420 according to the embodiment of the present invention may acquire an image corresponding to changes in real time when bending movement, anterior and posterior translation movement, pivot movement or the like is performed, and may store the acquired image in the database.

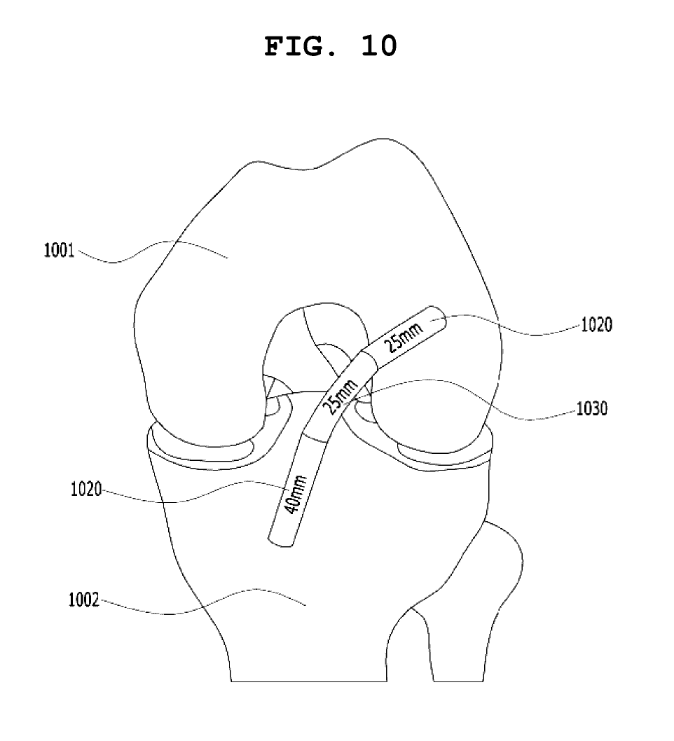

FIG. 10 is a view showing an example of the display of an inserted virtual ligament according to an embodiment of the present invention.

Referring to FIG. 10, the display control unit 460 or 540 may divide an inserted virtual ligament into a first portion 1010, a second portion 1020, and a third portion 1030 on a 3D model of a knee region, and may display them in different colors. In this case, the first portion 1010 refers to a virtual ligament portion inserted into a first bone 1001, a second portion 1020 refers to a virtual ligament portion inserted into a second bone 1002, and a third portion 1030 refers to a virtual ligament portion located between the first bone 1001 and the second bone 1002 and exposed to the outside.

By way of example, FIG. 10 shows an example in which the length of the first portion 1010 is 25 mm, the length of the second portion 1020 is 40 mm, and the length of the third portion 1030 exposed to the outside is 25 mm. Thereafter, changes in the lengths of the first to third portions may be determined by performing simulation using an image corresponding to changes in the movement of a knee region.

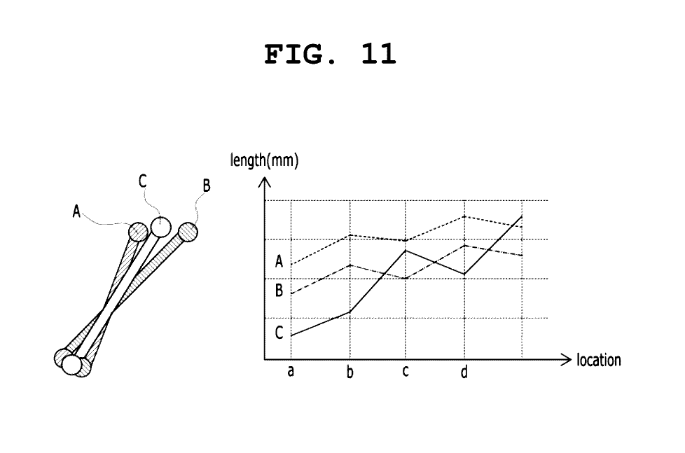

FIG. 11 is a view showing an example of the display of a graph representative of changes in the length of a ligament according to an embodiment of the present invention.

Referring to FIG. 11, the display control unit 460 or 540 may display changes in the length of a virtual ligament based on the location of the insertion of the virtual ligament received from a user in the form of a graph.

The horizontal axis of the graph may represent simulation case information based on the location of the insertion of the virtual ligament received from a user. As an example, “a” may represent the results of simulation for a first insertion location case, “b” may represent the results of simulation for a second insertion location case, and “c” may represent the results of simulation for a third insertion location case. That is, a user may change the location of a virtual ligament in various manners, and may determine the results of simulation for each location using the system according to an embodiment of the present invention. The user may simulate changes in the length of an inserted virtual ligament for various locations, and, thus, may find the most appropriate location of insertion at which a change in the length of the virtual inserted ligament is minimized.

Meanwhile, the vertical axis of the graph may represent information about the length of the ligament in millimeters (mm). As an example, “A” represents a curve representative of changes in the length of a first portion, “B” represents a curve representative of changes in the length of a second portion, and “C” represents a curve representative of changes in the length of a third portion.

In the example of FIG. 11, the lengths of the first to third portions have been changed least at location a at which simulation was performed first, and thus it can be seen that the most appropriate location into which an actual ligament will be inserted during the actual restorative surgery of a ligament is location a. In this case, the virtual ligament may include 3D coordinate information based on a 3D model of a knee region as insertion location information.

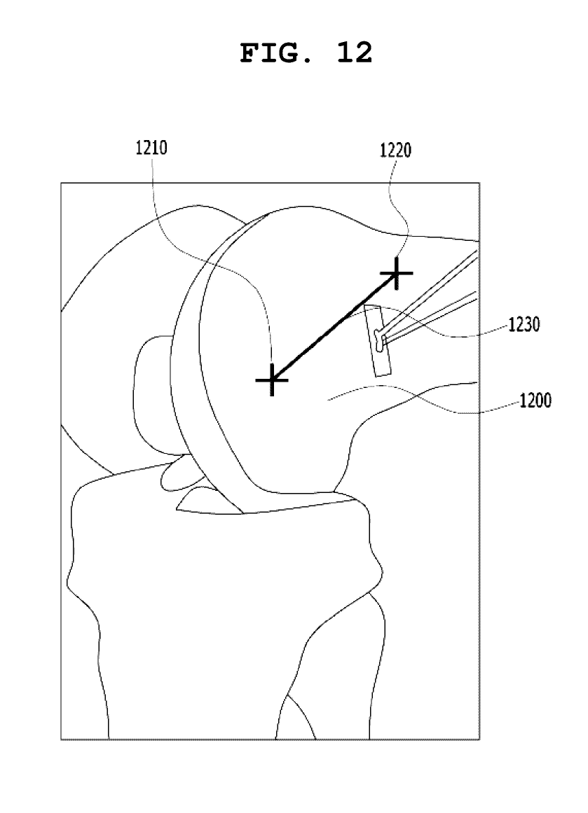

FIG. 12 is a view showing an example of the location of the insertion of a virtual ligament received from a user according to an embodiment of the present invention.

According to the embodiment of the present invention, the interface control unit 520 may receive the location of the insertion of a virtual ligament from a user. In this case, the interface control unit 520 may receive the start and end points of a ligament to be inserted into a first bone or a second bone as the location of the insertion of the virtual ligament. That is, the interface control unit 520 may receive the start and end points of a first portion and the start and end points of a second portion via mouse clicks. In this case, the location of a third portion may be automatically determined based on the start point or end point for the first and second portions.

Referring to FIG. 12, it can be seen that the start point 1210 and end point 1220 of a first portion 1230 have been input into a first bone 1200 by the interface control unit 520.

An operation flowchart according to an embodiment of the present invention is described in brief based on the above-described details.

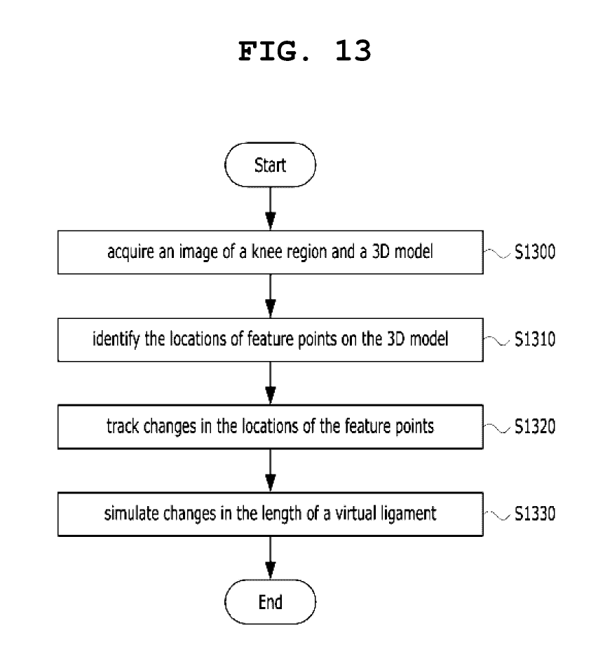

FIG. 13 is a first operation flowchart of a method for simulating the reconstructive surgery of a cruciate ligament using medical images according to an embodiment of the present invention.

Referring to FIG. 13, first, the method for simulating the reconstructive surgery of a cruciate ligament using medical images according to the embodiment of the present invention includes step S1300 of acquiring, by the image acquisition unit 320 or 420, an image of a knee region of a subject to which the restorative surgery of a ligament will be performed, and a 3D model which is generated to include the knee region.

Thereafter, the method according to the embodiment of the present invention including step S1310 of identifying, by the identification unit 330 or 430, the locations of a plurality of reference points, i.e., references for tracking changes in the movement of the knee region, on the 3D model (in particular, on a bone, or a rigid body region).

In this case, at step S1310, the reference points may be identified based on marking information input onto the 3D model by a user, or may be automatically extracted using image processing. Furthermore, at step S1310, the locations of the reference points may be primarily identified in a second image corresponding to the changes in the movement via markers actually attached or installed onto a knee bone or skin of a patient, and locations corresponding to the locations of the reference points identified in the second image may be identified on the 3D model by registering the second image with the 3D model. Since this has been described in detail above, a description thereof is omitted below.

Thereafter, the method according to the embodiment of the present invention includes step S1320 of acquiring, by the tracking unit 340 or 440, an image (i.e., a moving image) corresponding to the changes in the movement of the knee region attributable the movement, and tracking, by the tracking unit 340 or 440, changes in the locations of the reference points identified on the 3D model using the acquired image corresponding to the changes in the movement.

In this case, in an embodiment of the present invention, in order to acquire the image corresponding to the changes in the movement of the knee region via the tracking unit 340 or 440, a process in which a skilled doctor or the patient himself or herself actually moves a knee (i.e., the skilled doctor or patient subjects the knee to bending movement, anterior and posterior translation movement, pivot movement or the like) may be performed after step S1310. Furthermore, the tracking unit 340 or 440 tracks the changes in the locations of the reference points based on an image (i.e., a moving image) acquired via the process of moving the knee.

Thereafter, the method according to the embodiment of the present invention includes step S1330 of simulating, by the simulation unit 350, 450 or 530, changes in the length of a virtual ligament attributable to the changes in the movement of the knee region based on the changes in the locations of the reference points tracked at step S1320 and based on the location of the insertion of the virtual ligament received from the user.

In this case, the location of the insertion of the virtual ligament may receive from the user via the interface control unit 520.

Thereafter, although not shown in the drawing, the method according to the embodiment of the present invention may include the step of displaying, by the display control unit 460 or 540, the results of the simulation performed at step S1330 (i.e., the results of the changes in the length of the virtual ligament) on a screen. In this case, the display control unit 460 or 540 may display the results of the changes in the length of the virtual ligament in the form of a graph, may display first to third portions in different colors, and may display the location of the insertion of the virtual ligament, at which the results of the changes in the length of the virtual ligament attributable to the simulation is minimized, as a recommended insertion location.

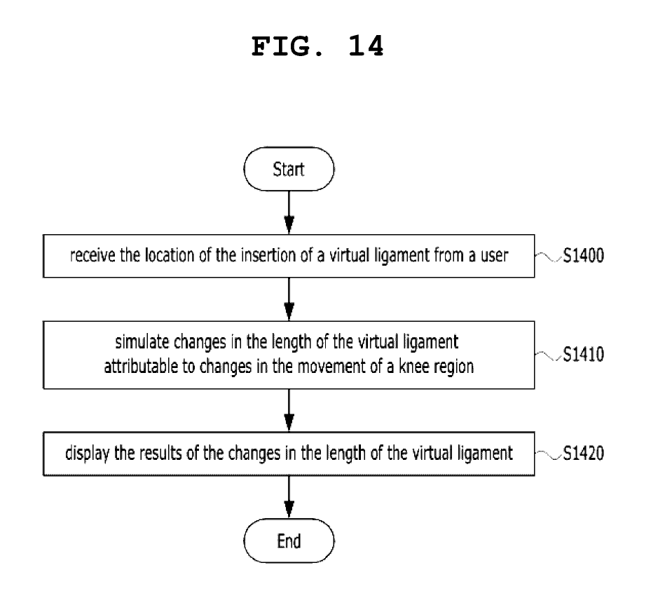

FIG. 14 is a second operation flowchart of a method for simulating the reconstructive surgery of a cruciate ligament using medical images according to an embodiment of the present invention.

Referring to FIG. 14, first, the method according to the embodiment of the present invention includes step S1400 of receiving, by the interface control unit 520, the location of the insertion of a virtual ligament from a user based on a previously stored image corresponding to changes in the movement of a knee region of a subject on which the restorative surgery of a ligament will be performed.

In this case, information about the previously stored image may include not only the image (i.e., a moving image) corresponding to the changes in the movement of the knee region of the subject on which the restorative surgery of a ligament will be performed, but also an image (a 2D image) of the knee region and an image of a 3D model generated to include the knee region.

Furthermore, at step S1400, the interface control unit 520 may receive the location of the insertion of the virtual ligament relative to a bone region or a rigid body region on a previously stored 3D model of the knee region as a plurality of reference points, i.e., references for tracking the changes in the length of the virtual ligament attributable to the changes in the movement of the knee region.

Thereafter, the method according to the embodiment of the present invention include step S1410 of simulating, by the simulation unit 350, 450 or 530, the changes in the length of the virtual ligament attributable to the changes in the movement of the knee region based on the input of the user received at step S1400.

In this case, the simulation unit 350, 450 or 530 may simulate changes in the length of each of the first to third portions.

Thereafter, the method according to the embodiment of the present invention includes step S1420 of displaying, by the display control unit 460 or 540, the results of the changes in the length of the virtual ligament attributable to the simulation.

In this case, the display control unit 460 or 540 may display a graph representative of changes in the length of each of the first portion to third portions of the virtual ligament as the results of the simulation. Furthermore, the display control unit 460 or 540 may display the first to third portions on the 3D model in different colors so that the user can easily identify the extent of changes in the length of each of the first to third portions. Since this has been described in detail above, a description thereof is omitted below.

Furthermore, the display control unit 460 or 540 may display a list of recommended insertion locations calculated based on the results of the changes in the length of the virtual ligament attributable to the simulation. Furthermore, the display control unit 460 or 540 may display numerical value information regarding the recommended insertion locations on a graph in an overlay manner.

The method for simulating the reconstructive surgery of a cruciate ligament using medical images according to an embodiment of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

The present invention has the advantage of providing a system and method for simulating surgery using medical images, particularly a system and method that are capable of simulating the restorative surgery of a cruciate ligament.

The present invention has the advantage of simulating changes in the length of a cruciate ligament when a knee moves, thereby reducing the occurrence rate of recurrence, instability, a reconstruction failure and the like that occur after the reconstructive treatment of a cruciate ligament.

The present invention has the advantage of providing the most appropriate location of the insertion of a ligament by taking into account the state of knee movement of a patient (i.e., a difference in the extent to which an implanted ligament is increased or decreased when a knee is flexed or extended or when the knee is rotated, a difference in tension, and the like).

The present invention has the advantage of acquiring information about the actual movement of a knee of a patient based on a medical image acquired by photographing the actual movement of the knee of the patient and then simulating changes in the length of a cruciate ligament attributable to the actual movement of the knee of the patient.

The present invention has the advantage of providing convenience to the restorative surgery of a cruciate ligament.

The present invention has the advantage of identifying the location of a reference point, i.e., a reference for tracking changes in the movement of a knee region, on a 3D model, acquiring an image corresponding to changes in the movement of the knee region, tracking changes in the location of the reference point identified on the 3D model using the acquired image corresponding to the changes in the movement, and simulating changes in the length of a virtual ligament attributable to the changes in the movement of the knee region based on the tracked changes in the location of the reference points and based on the location of the insertion of the virtual ligament received from a user, thereby virtually simulating changes in the length of the ligament during the period before and after surgery.

The present invention has the advantage of virtually simulating the restorative surgery of a cruciate ligament, thereby recommending the location of insertion, at which a change in the length of a ligament is minimized, as the most appropriate location of the insertion of the ligament.

In particular, the present invention has the advantage of simulating the interval between the femur and tibia of a patient attributable to the actual movement of a knee and, thus, simulating changes in the length and tension of a virtual cruciate ligament at the virtual location of the insertion of a cruciate ligament.

The present invention has the advantage of displaying a graph representative of changes in the length of each of a first portion inserted into a first bone, a second portion inserted into a second bone, and a third portion located between the first and second portions based on the first and second bones, which are included in a virtual ligament based on the location of the insertion of the virtual ligament received from a user, and also displaying the first to third portions in different colors, thereby providing changes in the length of a ligament in an intuitively and easily understandable form.

While the present invention has been described in conjunction with specific details, such as specific elements, and limited embodiments and diagrams, above, these are provided merely to help an overall understanding of the present invention. The present invention is not limited to these embodiments, and various modifications and variations can be made based on the foregoing description by those having ordinary knowledge in the art to which the present invention pertains.

Therefore, the technical spirit of the present invention should not be determined based only on the described embodiments, and the following claims, all equivalents to the claims and equivalent modifications should be construed as falling within the scope of the spirit of the present invention.

What is claimed is:

1. A method for simulating reconstructive surgery of a cruciate ligament using medical images executed by a processor within a computing system, the method comprising:
acquiring, by the processor, a first image of a knee region of a subject on which ligament reconstructive surgery will be performed, and a 3D model which is generated to include the knee region;
identifying, by the processor, locations of reference points, which are references for tracking changes in movement of the knee region, on the 3D model;
acquiring, by the processor, a second image corresponding to the changes in the movement of the knee region;
tracking, by the processor, changes in the locations of the reference points, identified on the 3D model, using the acquired second image corresponding to the changes in the movement; and
simulating, by the processor, changes in length of a virtual ligament attributable to the changes in the movement of the knee region based on the tracked changes in the locations of the reference points and a location of insertion of the virtual ligament received from a user.

2. The method of claim 1, wherein the identifying comprises:
identifying, by the processor, first locations of the reference points in the acquired second image corresponding to the changes in the movement; and
identifying, by the processor, the locations of the reference points on the 3D model corresponding to the first locations of the reference points by registering the second image with the 3D model.

3. The method of claim 1, wherein the identifying comprises identifying the locations of the reference points by extracting the locations of feature points relative to a bone region or a rigid body region on the 3D model, or identifying the locations of the reference points based on marking information received from the user.

4. The method of claim 1, further comprising displaying, by the processor, the changes in the length of the virtual ligament attributable to the simulation.

5. The method of claim 4, wherein the displaying comprises:
visualizing, by the processor, changes in length of a first portion of the virtual ligament attributable to the changes in the movement of the knee region, wherein the first portion is inserted into a first bone;
visualizing, by the processor, changes in length of a second portion of the virtual ligament attributable to the changes in the movement of the knee region, wherein the second portion is inserted into a second bone; and
visualizing, by the processor, changes in length of a third portion of the virtual ligament attributable to the changes in the movement of the knee region, wherein the third portion is located between the first and second portions.

6. The method of claim 1, further comprising:
displaying, by the processor, a first portion of the virtual ligament, inserted into a first bone, in a first color;
displaying, by the processor, a second portion of the virtual ligament, inserted into a second bone, in a second color; and
displaying, by the processor, a third portion of the virtual ligament, located between the first and second portions, in a third color.

7. The method of claim 4, further comprising displaying the location of the insertion of the virtual ligament, at which the changes in the length of the virtual ligament attributable to the simulation are minimized, as a recommended insertion location.

8. A method for simulating reconstructive surgery of a cruciate ligament using medical images executed by a processor within a computing system, the method comprising:
receiving, by the processor, at least one location of insertion of a virtual ligament from a user on a previously stored image corresponding to changes in movement of a knee region of a subject on which ligament reconstructive surgery will be performed;
simulating, by the processor, changes in length of the virtual ligament attributable to the changes in the movement of the knee region based on the at least one location of the insertion of the virtual ligament received from the user; and
displaying, by the processor, the changes in the length of the virtual ligament attributable to the at least one location of the insertion of the virtual ligament received from the user using the results of the simulation.

9. The method of claim 8, wherein the receiving comprises receiving the location of the insertion of the virtual ligament relative to a bone region or a rigid body region on a previously stored 3D model of the knee region as locations of reference points, which are references for tracking the changes in the length of the virtual ligament attributable to the changes in the movement of the knee region.

10. The method of claim 8, wherein the displaying comprises:
visualizing, by the processor, changes in length of a first portion of the virtual ligament attributable to the at least one location of the insertion of the virtual ligament, wherein the first portion is inserted into a first bone;
visualizing, by the processor, changes in length of a second portion of the virtual ligament attributable to the at least one location of the insertion of the virtual ligament, wherein the second portion is inserted into a second bone; and
visualizing, by the processor, changes in length a third portion of the virtual ligament attributable to the at least one location of the insertion of the virtual ligament, wherein the is located between the first and second portions.

11. The method of claim 8, further comprising:
displaying, by the processor, a first portion of the virtual ligament, inserted into a first bone, in a first color;
displaying, by the processor, a second portion of the virtual ligament, inserted into a second bone, in a second color; and
displaying, by the processor, a third portion of the virtual ligament, located between the first and second portions, in a third color.

12. The method of claim 8, further comprising displaying, by the processor, a list of recommended insertion locations calculated based on the simulation results of the changes in the length of the virtual ligament attributable to the at least one location of the insertion of the virtual ligament received from the user.

13. A system for simulating reconstructive surgery of a cruciate ligament using medical images, the system comprising a processor, the processor is configured to:
acquire a first image of a knee region of a subject on which ligament reconstructive surgery will be performed, and a 3D model which is generated to include the knee region;
identify locations of reference points, which are references for tracking changes in movement of the knee region, on the 3D model;
acquire a second image corresponding to the changes in the movement of the knee region;
track changes in the locations of the reference points, identified on the 3D model, using the acquired second image corresponding to the changes in the movement; and
simulate changes in length of a virtual ligament attributable to the changes in the movement of the knee region based on the tracked changes in the locations of the reference points and a location of insertion of the virtual ligament received from a user.

14. The system of claim 13, wherein the processor is further configured to:
identify first locations of the reference points in the acquired second image corresponding to the changes in the movement; and
then identify the locations of the reference points on the 3D model corresponding to the locations of the reference points by registering the second image with the 3D model.

15. The system of claim 13, wherein the processor is further configured to:
identify the locations of the reference points by extracting the locations of feature points relative to a bone region or a rigid body region on the 3D model or identify the locations of the reference points based on marking information received from the user.

16. The system of claim 13, wherein the processor is further configured to display the changes in the length of the virtual ligament attributable to the simulation.

17. The system of claim 16, wherein the processor is further configured to display the location of the insertion of the virtual ligament, at which the changes in the length of the virtual ligament attributable to the simulation are minimized, as a recommended insertion location.

18. A system for simulating reconstructive surgery of a cruciate ligament using medical images, the system comprising a processor, the processor is configured to:
receive at least one location of insertion of a virtual ligament from a user on a previously stored image corresponding to changes in movement of a knee region of a subject on which ligament reconstructive surgery will be performed;
simulate changes in length of the virtual ligament attributable to the changes in the movement of the knee region based on the at least one location of the insertion of the virtual ligament received from the user; and
display the changes in the length of the virtual ligament attributable to the at least one location of the insertion of the virtual ligament received from the user using the results of the simulation.

19. The system of claim 18, wherein the processor is further configured to receive the location of the insertion of the virtual ligament relative to a bone region or a rigid body region on a previously stored 3D model of the knee region as locations of reference points, which are references for tracking the changes in the length of the virtual ligament attributable to the changes in the movement of the knee region.

20. The system of claim 18, wherein the processor is further configured to display a list of recommended insertion locations calculated based on the simulation results of the changes in the length of the virtual ligament attributable to the at least one location of the insertion of the virtual ligament received from the user.
